(19) United States
(12) Patent Application Publication
(10) Pub. No.: US 2026/0069896 A1
(43) Pub. Date: Mar. 12, 2026

(54) ULTRASONIC TREATMENT OF VITREOUS OPACITIES

(71) Applicants: SUNNYBROOK RESEARCH INSTITUTE, TORONTO (CA); VITREOSONIC INC, TORONTO (CA)

(72) Inventors: GARY LLOYD KA TAO YAU, NORTH YORK (CA); KULLERVO HENRIK HYNYNEN, TORONTO (CA)

(21) Appl. No.: 19/277,566

(22) Filed: Jul. 23, 2025

Related U.S. Application Data
(63) Continuation of application No. 18/167,602, filed on Feb. 10, 2023, now Pat. No. 12,390,667.
Application No. 18/167,602 is a continuation of application No. PCT/US2021/046819, filed on Aug. 20, 2021.
(60) Provisional application No. 63/068,643, filed on Aug. 21, 2020.

(51) Int. Cl. A61N 7/02; A61N 7/00

(52) U.S. Cl. CPC A61N 7/02; A61N 2007/0052

(57) ABSTRACT

In at least some examples, a method of treating an eye, includes (i) imaging a patient's vitreous using a probe, (ii) defining a window on a desired region of the vitreous, (iii) administering ultrasonic energy treatment to the desired region within the window, (iv) continually monitoring the treatment via the probe, (v) adjusting a characteristic of the ultrasonic energy treatment based on the monitoring, (vi) re-imaging the desired region of the vitreous after the treatment is administered, and (vii) evaluating or observing the desired region to determine whether a target percentage of a vitreous opacity has been resolved.

### CROSS-REFERENCE TO RELATED APPLICATIONS

This application is a continuation of International Application No. PCT/US21/46819, filed Aug. 20, 2021, which claims priority to U.S. Provisional Application Serial No. 63/068,643, filed Aug. 21, 2020, the disclosure of which is incorporated by reference as if fully set forth herein.

### FIELD OF THE DISCLOSURE

The present disclosure relates to devices, systems, and methods for treating vitreous opacities. More particularly, the present disclosure relates to devices,, systems, and methods for using ultrasonic waves for treating vitreous opacities.

### BACKGROUND OF THE DISCLOSURE

Vitreous hemorrhage (VH) is a common ocular blinding condition. Patients suffering from vitreous hemorrhages that do not naturally resolve are treated surgically. Common surgical methods are invasive and uncomfortable for the patient. Additionally, they may result in infection. Surgical methods are also invasive, so have with them inherent risks, including retinal detachment and infection, both of which if they were to occur, are severe complications that can lead to permanent blindness. Furthermore, currently all vitreous surgery is performed in operating rooms, which increases the cost of the treatment.

Thus, it would be beneficial to develop new non-invasive techniques and devices to treat vitreous hemorrhage.

### SUMMARY OF THE DISCLOSURE

In at least some examples, a method of treating an eye, includes (i) imaging a patient's vitreous using a probe, (ii) defining a window on a desired region of the vitreous, (iii) administering ultrasonic energy treatment to the desired region within the window, (iv) continually monitoring the treatment via the probe, (v) adjusting a characteristic of the ultrasonic energy treatment based on the monitoring, (vi) re-imaging the desired region of the vitreous after the treatment is administered, and (vii) evaluating or observing the desired region to determine whether a target percentage of a vitreous opacity has been resolved.

### BRIEF DESCRIPTION OF THE DISCLOSURE

Various embodiments of the presently disclosed devices, systems and methods are shown herein with reference to the drawings, wherein:

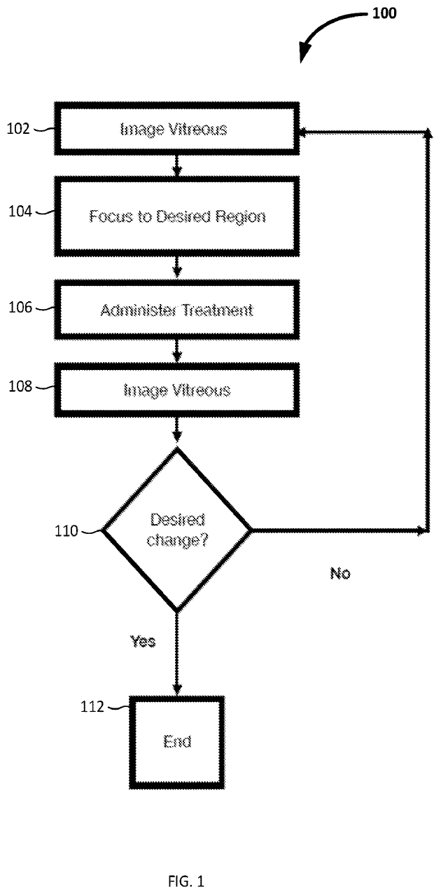

FIG. 1 is schematic workflow diagram of a method of treating vitreous hemorrhage;

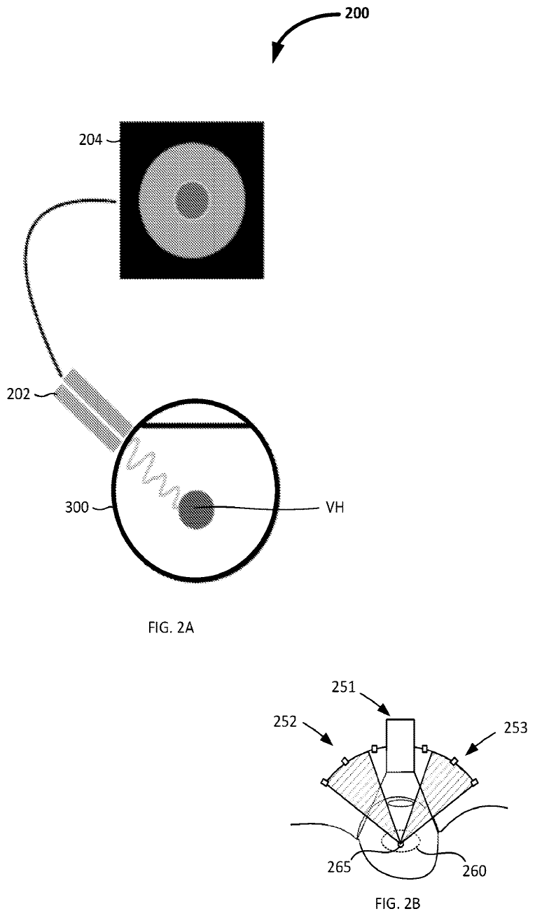

FIGS. 2A-B are schematic diagram showing components of an ultrasonic device according one embodiment of the present disclosure; and

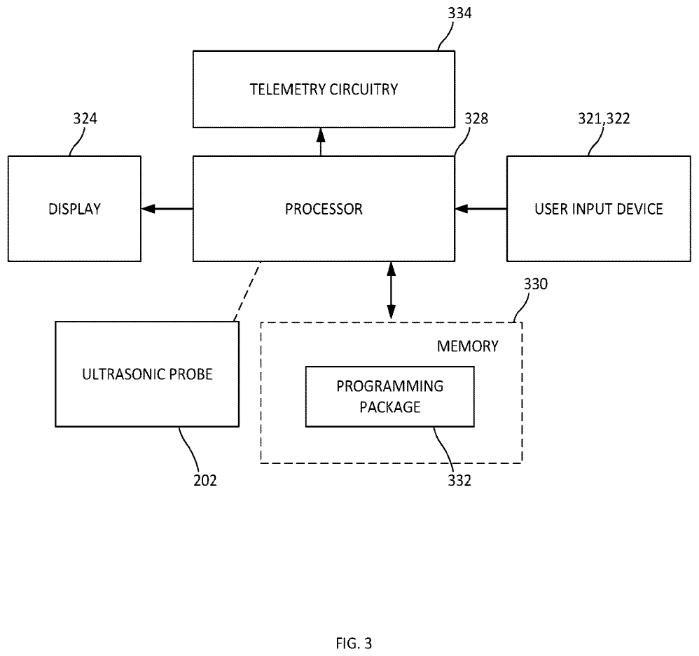

FIG. 3 is a block diagram of the components of a system according to the present disclosure.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to systems and methods of treating vitreous hemorrhage, conventional techniques and devices suffer from some shortcomings as described above.

There therefore is a need for further improvements to the devices, systems, and methods of treating vitreous hemorrhage. Among other advantages, the present disclosure may address one or more of these needs.

As used herein, the term “proximal,” when used in connection with a component of a probe assembly, refers to the end of the component closest to the physician when the probe is used on a patient, whereas the term “distal,” when used in connection with a component of a probe assembly, refers to the end of the component farthest from the physician when the assembly is used on a patient.

Likewise, the terms “trailing” and “leading” are to be taken as relative to the operator (e.g., physician) of the probe assembly. “Trailing” is to be understood as relatively close to the operator, and “leading” is to be understood as relatively farther away from the operator.

It is believed that the rate of vitreous hemorrhage clearance/resorption from the eye can be accelerated by hemolysis, fibrinolysis, increase in local phagocytic activity, and vitreous liquefaction (which involves a breakdown of collagen fibres in the vitreous macrostructure).

In at least some examples, ultrasonic waves may be used in a non-invasive technique to affect at least some of these factors via thermal and/or non-thermal mechanisms, potentially making it a viable treatment modality to accelerate vitreous hemorrhage clearance.

The present systems, method and techniques improve on previous techniques and methods. Specifically, the present disclosure includes an image-guided ultrasonic treatment system for vitreous hemorrhage. In one example, the system contains the ability to modify treatment parameters, combined with a concurrent imaging system providing structural and/or functional information at the time of treatment that can enhance the success of the treatment. For example, as shown in FIG. 1, a method for treating vitreous hemorrhage 100 may include imaging the vitreous 102, focusing on a desired region of the vitreous 104, administering ultrasound non-invasively 106, re-imaging the vitreous after treatment 108, measuring or observing desired changes 110 and repeating the process if the desired change has not occurred, or ending the process 112 if the method has been successful in clearing up the vitreous hemorrhage. In some examples, this step includes structural/direct view, but aided with functional information, such as:
1. direct observation via ultrasound image of the clot;
2. functional measurement of real-time temperature;
3. functional measurement of elasticity; and/or
4. functional measurement of hemoglobin status.

FIG. 2A is a schematic diagram of an exemplary system. Ultrasonic system 200 may include a dual probe 202 that includes a transducer for therapeutic ultrasound probe (0.5 to 10 MHz) and a diagnostic ultrasound probe (5 to 20 MHz) and may include an optical or infrared camera. Probe 202, which can be handheld or fixed in a manual or motorized positioning device, may be applied to a non-transparent portion of the eye 300 (e.g., the sclera), so as to limit exposure of the ultrasonic waves to the cornea and lens. The optical/infrared camera visualizes the eye through the lens. In some embodiments the ultrasound imaging may be done through the cornea and the lens and in some embodiments the optical imaging is done via optical fiber to minimize the size of the probe. In some embodiments the probe 202 will have one or more ultrasound receivers to detect the scattered ultrasound signals that can be used to control the exposure and/or to monitor the safety and/or to detect the photoacoustic signals. In some embodiments the probe will include an optical fiber to deliver laser pulses into the eye for photoacoustic imaging. In some embodiments the therapeutic ultrasound transducer is a single or multi element fixed focus transducer. The focusing is achieved by overlapping fields, lenses, reflectors, variable path propagation device, or making the transducer curved such as to achieve the desired acoustic focus. In some embodiments the therapy transducer is a phased array that can perform electronic focusing and beam steering. The probe 202 may be in communication with a viewing system 204 for the operator, which displays the corresponding image of the eye. In some examples, the probe 202 is wired to viewing system 204. In at least some other examples, dual probe 202 is wirelessly connected to viewing system 204 via WLAN, Bluetooth or other suitable communications protocol. Once a target is identified on the screen, which corresponds to the vitreous hemorrhage “VH” in the eye, the operator can then initiate the treatment. This process is repeated until the therapeutic endpoints are achieved as described in the process of FIG. 1. In FIG. 2B, an exemplary system is shown of an integrated probe that includes an imaging element 251, ultrasonic detectors 252 and a therapy beam 253. The integrated probe may be disposed and oriented toward a target 260, and the treatment and diagnostic elements may be focused at a certain position 265.

There are at least four ways how ultrasound can be used for the treatment of the hemorrhage. First, it will be appreciated that elevated temperatures can cause hemolysis. Therefore, the highly focused ultrasound can be used to cause a quick, local temperature elevation above the threshold of the cell membrane rupture. Second, high pressure amplitude ultrasound bursts can cause gas bubble formation at the focus. This is called cavitation. The collapse is associated with a shock wave that can disintegrate the cells. Third, longer high-pressure amplitude burst either with or without cavitation can cause the increase the tissue temperature above the water boiling point and cause vaporization. Fourth, ultrasound absorption in medium causes an energy loss from the beam that translates into a radiation force. This radiation force can be used to move fluid or cells noninvasively. Therefore, the radiation force could be used to disperse the clot. Each of these bio-effects can be used either alone or in combination with one or more of the other bio-effects for the treatments of the Vitreous hemorrhages. Vitreous hemorrhage presents in a heterogeneous manner, and hence differing regions require a customized treatment, even within the same eye. For example, thicker clotted blood may require a short pulse of high intensity focused ultrasound to achieve a certain temperature and subsequent ablation to a specified focal region, followed immediately with moderate intensity pulsed radiation force-based treatment to mechanically disperse the clot. Conversely, diffuse non-clotted red blood cells homogeneously mixed within a more liquefied vitreous may be treated with moderate intensity over a longer period over a wider volume, to efficiently elevate the temperature of a wider volume of vitreous to allow for hemolysis, while relying on milder non-thermal mechanisms such as radiation force induced steaming to induce desired changes.

To increase the specificity of the delivery of ultrasonic energy for treatment, a device may be configured to manipulate any one, two, three or four of the parameters described above (i.e., local temperature rise, cavitation, vaporization, radiation and force). Any combination of these parameters may be used to adjust performance of an ultrasonic probe in direct response to the real time monitoring data feedback. In some examples, a focused ultrasound device may choose any of the four parameters, or combination thereof, (e.g., based on a strength or magnitude of the parameter) and adjust the performance of the therapeutic probe accordingly. A therapeutic device may also make adjustments to the performance based on all four, or any combination of the four parameters by utilizing a weighted average or other suitable weighting method. Additionally, to allow for these customized treatments, the current system may allow for real-time adjustments in acoustic output, level of beam focusing, and/or pulsed or continuous treatments depending on the goals of therapy.

At ultrasound intensities below the cavitation threshold of the vitreous and blood cells (e.g., between 0.1 W/cm² and 3.5 W/cm²), relative hyperthermia can induce temperature elevations that result in hemolysis as well as liquefy vitreous, both of which can accelerate clearance. At high intensities (i.e., >800 W/cm²), with temperatures reaching >100 deg C, the target region may be vaporized. At intensities above the cavitation intensities short ultrasound bursts can result in mechanical fragmentation of the clot. In at least some examples, the present systems, devices and methods will allow for all of these modes of therapies.

Additionally, dynamic electronic or mechanical beam focusing allows for accurate targeting of the region of interest. Without being bound by any particular theory, it is believed that advances in ultrasound transducers allow for dynamic steering that can change the focal point, and hence allow for robust and efficient changes in focusing. This may be advantageous as different regions in the vitreous cavity can then be targeted quickly and in real-time. As an added feature, a computer/processor can “lock-in” to this region on the diagnostic ultrasound to assist the user. This region can be a focal point, and/or a volume. With respect to a volume treatment, the focal area may be defocused enough so as to expand the focal region up to a certain volume.

In some examples, a “volume” treatment may be performed by having the computer lock in to a region, and then subsequent focal and focused treatments in rapid succession within that volume, with the pulses being so short in between that it is seemingly simultaneous or the sonications may be separated by a time interval to allow tissue cooling prior the next sonication. This range can include focal treatment areas or volumetric which can encompass the entire vitreous body.

Using the disclosed method and techniques, the effects may be contained to the region of interest, and not cause any significant biologic effect to non-targeted adjacent tissues. In some examples, the user may manually avoid the non-targeted tissue. Additionally, because the vitreous body has a very distinct imaging characteristic from surrounding features, a computer algorithm may serve as an automatic differentiator as an additional failsafe.

In some examples, continuous treatments will allow for an accumulation of absorption of insonified energy, elevating temperature of the target tissue. Conversely, pulsed treatments can limit temperature accumulation, and hence may be more appropriate for inducing non-thermal effects. Again, this setting will be able to be manipulated in real-time depending on the characteristics of the target region.

To facilitate this customized treatment, an equally advanced diagnostic ultrasound component may accompany the treatment unit. This real-time imaging unit may allow for treatment planning, real time image-guidance and or control during treatment, and visualization of effect on the insonified region.

In addition to the structural changes seen during treatment, including the visualization of the movement of the vitreous, as well as any hypo- or hyper-echoic regions as a result of treatment (which signifies a biologic mechanical effect or cavitation), three additional functional parameters may further assist the operator during the treatment and for subsequent monitoring of progress.

The first functional parameter may include thermography, which includes providing an estimate of the spatial temperature distribution of the insonified region. This can be measured via ultrasound thermography. For this purpose the temperature dependent ultrasound scattering and/or speed of sound or tissue stiffness may be calibrated for temperature estimation.

The second functional parameter is a measure of hemoglobin level and status. Photoacoustic imaging, where a laser light is emitted at a frequency, which is specific to hemoglobin absorption, causing local temperature elevation and thermal expansion that induces an ultrasound signal that can be detected by the receivers in the probe. These signals may be localized and may be used to quantify hemoglobin in the vitreous cavity. Hemoglobin has a specific electromagnetic radiation absorption signature that can be identified via photoacoustic imaging. Further, hemoglobin variants (oxy-hemoglobin and deoxy-hemoglobin) also have distinct electromagnetic absorption characteristics, and the quantification of this can also inform the stage of vitreous hemorrhage. In addition to a real-time display of this information, this measure can provide a means to monitor and/or control effects with sequential therapy.

The third functional parameter is elastography, which is a measure of the stiffness within the vitreous body. The vitreous, particularly with hemorrhage, is a heterogenous space, and presumably varying elasticity of the vitreous opacity may warrant specific treatment requirements. The stiffness changes can be monitored for example by using local harmonic motions methods where the therapy beam is pulsed and the radiation force induced tissue displacement tracked by ultrasound between the short (for example 50 ms) bursts.

To allow the clinician to perform these functions, a system 300 may include additional components such as a mouse 321, a keyboard 322, and a display screen 324. It is to be understood that in addition to, or in lieu of, the mouse 321, other directional programming devices may be used, such as a joystick, touch screen or directional keys included as part of the keys associated with the keyboard 322. As shown in FIG. 3, the system generally includes a processor 328 (e.g., a central processor unit (CPU) ) in communication with ultrasonic probe 202 and memory 330 that stores a programming package 332, which can be executed by the processor 328 to allow a clinician to program probe. The system 300 may further include telemetry circuitry 334 for downloading treatment parameters to the probe and uploading treatment data or information to another computer or network. The telemetry circuitry 334 may also be configured for transmitting the control data and receiving status information.

Thus, the described system may provide a non-invasive medical device with the goal of accelerating vitreous hemorrhage clearance. It consists of a customizable platform for therapeutic ultrasound, in combination with real-time ultrasound based diagnostic system providing structural and functional feedback to optimize and/or control the treatment. Additionally, the same or similar systems, methods or devices may be used to diagnose, detect, and/or treat non-hemorrhage opacities or other vitreous opacities. Though the description above has been primarily directed to clearing blood in the eye, this is only exemplary and other methods of treatment of the eye are also contemplated. For example, certain vitreous “floaters,” are an aggregation of vitreous macromolecules that are suspended within the vitreous body, which can result in visually disabling symptoms. These vitreous opacities can be resolved using these techniques as well. Thus, the present devices and methods may be used to treat not only vitreous hemorrhage, but also other vision disturbing vitreous opacities.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1-20. (canceled)

21. A system for treating a vitreous hemorrhage in an eye using real-time ultrasound image guidance, the system comprising:
an ultrasound probe; and
control and processing circuitry operably connected to the ultrasound probe, the control and processing circuitry comprising a processor and memory, the memory storing instructions executable by the processor for performing operations comprising:
controlling the ultrasound probe to obtain one or more initial images of the eye;
processing the one or more initial images to detect, within a vitreous of the eye, an opacity associated with a vitreous hemorrhage;
performing real-time image-guided ultrasound treatment to deliver and control treatment to the opacity, the real-time image-guided ultrasound treatment comprising:
controlling the ultrasound probe to deliver focused ultrasound energy to a region within the vitreous and acquire real-time ultrasound images to facilitate monitoring of the treatment, the region comprising the vitreous hemorrhage;
controlling the ultrasound probe such that the ultrasound energy is delivered with sufficiently high pressure amplitude to induce cavitation within the region, the presence of cavitation being detected based on ultrasound signals scattered by gas bubbles within the region;
processing the real-time ultrasound images to monitor cavitation-induced disintegration or fragmentation of the vitreous hemorrhage; and
continuing the real-time image-guided ultrasound treatment until a therapeutic endpoint associated with sufficiency of clearance of the vitreous hemorrhage has been achieved.

22. The system according to claim 21 wherein the control and processing circuitry is configured to employ the real-time ultrasound images to adjust a characteristic of the focused ultrasound energy.

23. The system according to claim 21 wherein the control and processing circuitry is configured to employ the real-time ultrasound images to perform real-time adjustments in at least one of acoustic output and a level of beam focusing.

24. The system according to claim 21 wherein the control and processing circuitry is configured to employ the real-time ultrasound images to perform real-time adjustments to switch between pulsed treatment and continuous treatment.

25. The system according to claim 21 wherein the control and processing circuitry is configured to employ the real-time ultrasound images to generate, in real-time, structural information associated with the vitreous hemorrhage, and wherein the ultrasound probe is also controlled by the control and processing circuitry to generate, in real-time, functional information associated with treatment of the vitreous hemorrhage, and wherein the structural information and the functional information are employed to adjust a characteristic of the focused ultrasound energy.

26. The system according to claim 21 further comprising, prior to delivering the focused ultrasound energy to the region, processing the one or more initial images and employing distinct imaging characteristics of the vitreous to identify the vitreous from surrounding features of the eye; and
employing the identified vitreous to detect the opacity associated with the vitreous hemorrhage.

27. The system according to claim 21 wherein the ultrasound probe is controlled by the control and processing circuitry to generate a sufficiently high temperature within the region to cause hemolysis.

28. The system according to claim 27 wherein the control and processing circuitry is configured to process the real-time ultrasound images to determine when the hemolysis is induced.

29. The system according to claim 21 wherein the ultrasound probe is controlled by the control and processing circuitry to generate a sufficiently high radiation force within the region to facilitate mechanical dispersion of the vitreous hemorrhage.

30. The system according to claim 21 wherein the ultrasound probe is controlled by the control and processing circuitry to effect liquefaction of the vitreous in the region for facilitating accelerated clearance of the vitreous hemorrhage.

31. The system according to claim 21 wherein the one or more initial images are processed to identify two or more regions within the vitreous, and wherein the one or more initial images are further processed to determine a customized treatment for each region of the two or more regions based on characteristics of the vitreous hemorrhage within each region, and wherein the ultrasound probe is controlled, by the control and processing circuitry, to provide real-time image-guided ultrasound treatment to each region according to the customized treatment for each region.

32. The system according to claim 21 wherein the ultrasound probe comprises an ultrasound array and wherein electronic focusing and beam steering are employed to deliver the focused ultrasound energy to the opacity associated with the vitreous hemorrhage.

33. The system according to claim 21 wherein the ultrasound probe comprises a fixed-focus ultrasound transducer and wherein a motorized positioning device is employed to deliver the focused ultrasound energy to the opacity associated with the vitreous hemorrhage.

34. The system according to claim 21 wherein the focused ultrasound energy is delivered to a volume within the region by controlled focusing or defocusing of an ultrasound beam.

35. The system according to claim 21 wherein the focused ultrasound energy is delivered to a volume within the region by delivering a sequence of focused treatments within the volume to collectively treat the volume.

36. The system according to claim 35 wherein each focused treatment of the sequence of focused treatments is provided after a time interval selected to facilitate cooling of tissue between the focused treatments.

37. The system according to claim 21 wherein the focused ultrasound energy is delivered to the opacity associated with the vitreous hemorrhage after receiving input from an operator.

38. The system according to claim 37 wherein the input from the operator is received after displaying, on a viewing system, a guidance image identifying the opacity associated with the vitreous hemorrhage.

39. The system according to claim 1 wherein the therapeutic endpoint associated with sufficiency of clearance of the vitreous hemorrhage is evaluated by comparing a level of clearance of the vitreous hemorrhage to a pre-determined target level.

40. A method of treating a vitreous hemorrhage in an eye using real-time ultrasound image guidance, the method comprising:
employing an ultrasound probe to obtain one or more initial images of the eye;
processing the one or more initial images to detect, within a vitreous of the eye, an opacity associated with a vitreous hemorrhage;
performing real-time image-guided ultrasound treatment to deliver and control treatment to the opacity, the real-time image-guided ultrasound treatment comprising:
controlling the ultrasound probe to deliver focused ultrasound energy to a region within the vitreous and acquire real-time ultrasound images to facilitate monitoring of the treatment, the region comprising the vitreous hemorrhage;
controlling the ultrasound probe such that the ultrasound energy is delivered with sufficiently high pressure amplitude to induce cavitation within the region, the presence of cavitation being detected based on ultrasound signals scattered by gas bubbles within the region;
processing the real-time ultrasound images to monitor cavitation-induced disintegration or fragmentation of the vitreous hemorrhage; and
continuing the real-time image-guided ultrasound treatment until a therapeutic endpoint associated with sufficiency of clearance of the vitreous hemorrhage, evaluated based on the real-time ultrasound images, has been achieved.
